SEQUENCE LISTING (2 sequences)

SEQ ID NO: 1 - protein, 9 aa, Artificial Sequence
  example of an integrin binding peptide
  MISC_FEATURE (2)..(3)
  MISC_FEATURE (2)..(3): X = 8-amino-3,6-dioxaoctanoic acid N-terminal acetylation C-terminal amino group
CXXGRGDSP

SEQ ID NO: 2 - protein, 7 aa, Artificial Sequence
  example of an integrin binding peptide
GRGDSPK

(19) United States
(12) Patent Application Publication
(10) Pub. No.: US 2025/0099656 A1
(43) Pub. Date: Mar. 27, 2025

(54) Coated Medical Product and Method for Coating a Medical Product

(71) Applicant: phenox GmbH, Bochum (DE)

(72) Inventors: Tim LENZ-HABIJAN, Herne (DE); Catrin BANNEWITZ, Bochum (DE); Volker TRÖSKEN, Witten (DE); Ralf HANNES, Dortmund (DE); Hermann MONSTADT, Bochum (DE); Hans HENKES, Stuttgart (DE)

(73) Assignee: phenox GmbH, Bochum (DE)

(21) Appl. No.: 18/275,494

(22) PCT Filed: Feb. 2, 2022
(86) PCT No.: PCT/EP2022/052403

§ 371 Date: Aug. 2, 2023

(30) Foreign Application Priority Data
Feb. 2, 2021 (DE) DE102021102377.7

(51) Int. Cl. A61L 31/10; A61L 27/34; A61L 27/40; A61L 27/54; A61L 31/14

(52) U.S. Cl. CPC A61L 31/10; A61L 27/34; A61L 27/40; A61L 27/54; A61L 31/14; A61L 2300/412

(57) ABSTRACT

The invention relates to a medical product, with at least portions of the medical product being provided with a coating, said coating comprising a functional layer, and the functional layer containing saccharides and peptide sequences having integrin-binding motifs. The medical product proposed by the invention exhibits improved cell adherence and, at the same time, reduced thrombocyte adhesion and aggregation, which promotes ingrowth into the surrounding tissue, while the aggregation of thrombocytes and, in particular, restenosis are prevented.

The invention relates to a coating for medical products, wherein the coating features properties promoting cell adherence.

The use or application of medical products for the treatment of various disease patterns is constantly increasing. Many of these medical products are implants that enter the vascular system or the patient's body in general to permanently remain there during treatment.

To ensure that such an implant is not permanently perceived as a foreign body, it is advantageous if the implant grows in as soon as possible after implantation. For this purpose, the body's own cells must be caused to adhere to the implant. It is therefore advantageous to have an implant provided with a coating that promotes cell adherence. Moreover, the implant should not have any inflammatory potential. Restenosis must be prevented as well in the case of stents and similar medical products implanted in a blood vessel.

An amino acid sequence that promotes cell adhesion and mechanical anchorage of cells is the RGD sequence according to the one-letter code, which is composed of the amino acids arginine, glycine, and aspartic acid. This sequence occurs in proteins of the extracellular matrix, for example fibronectin and vitronectin. Cells are able to bind to this amino acid sequence with the help of integrins (cell surface receptors). Other peptide sequences with integrin-binding motifs are also known.

A stent having improved re-endothelialization properties is described in publication WO 2008/143933 A1. Although binding of integrin-selective peptides to integrins on the cell surface activates a signaling cascade that could induce proliferation of the cell and thus cause restenosis, the inventors found that despite integrin activation, the RGD peptide in fact counteracts restenosis. This is attributed to increased adhesion of endothelial cells to the stent, which ensures rapid endothelialization and ingrowth of the stent into the surrounding tissue, so that in turn the occurrence of restenosis is prevented.

Of crucial importance regarding implants placed in blood vessels is, however, the prevention of blood clotting triggered by the implant itself, i.e., the prevention of thrombocyte adhesion and thrombocyte aggregation. Such a phenomenon, and thus the formation of blood clotting or thrombi, can be observed with permanent or temporary implants as well as with medical instruments that are inserted into the patient's body only briefly for treatment or diagnostic purposes. The thrombocytes adhere to the surface of the introduced medical product marked by the body's endogenous proteins (thrombocyte adhesion), which as a result may lead to the formation of a thrombus (thrombocyte aggregation).

If such a thrombus is set free in the vascular system, for example, it can lead to secondary diseases, some of which can be serious or even fatal. This is primarily the case if the detached thrombus is washed away and lodges in the form of an embolus in smaller vessels, largely occluding them and as a consequence jeopardizing the sufficient perfusion of the affected areas. Resulting clinical pictures can be, for example, strokes, heart attacks or thromboses.

With a view to minimizing these risks for the patient, dual platelet inhibition is nowadays common practice as a rule to reduce the risk of thrombosis in the event surgery or interventions are necessary. In this case, the patient usually is given an initial dose of a combination of antiplatelet drugs/platelet aggregation inhibitors, in particular a combination of acetylsalicylic acid (ASA) and clopidogrel, before the surgery and must continue to take them regularly for a certain period of time after the procedure. Aside from this, other platelet aggregation inhibitors mentioned in the relevant guidelines on dual platelet inhibition may also be administered. These are usually applied instead of the clopidogrel in combination with ASA. Examples are prasugrel or ticagrelor.

The disadvantage of dual platelet inhibition is that it is systemic and therefore has also a systemic effect. Thus, for the duration of dual platelet inhibition, the patient's overall risk of bleeding is increased.

Another disadvantage is that dual platelet inhibition is not at all an option for some patient groups, for whom the risks associated with dual platelet inhibition, particularly the increased risk of bleeding, are so great that they definitely outweigh the benefits. In extreme cases, some patients may not even have access to certain treatment approaches simply because they cannot be adequately protected against the risk of clot formation after an intervention has taken place. For example, a dual platelet inhibition treatment is established practice adopted after the implantation of endovascular prostheses (stents and the like). For this reason, it has been proposed in publication WO 2018/210989 A1 to coat medical products with saccharides oligo- or polymerizing only when binding to the substrate. These mimic the natural glycocalyx, which covers the cells of blood vessels with a kind of mucus layer and consists of various polysaccharides covalently bound to membrane proteins (glycoproteins) and membrane lipids (glycolipids). This is therefore a biomimetic effect.

It is therefore the objective of the invention to provide medical products that ensure rapid ingrowth of the medical product into the surrounding tissue by enhancing cell adhesion or cell adherence, but at the same time preventing increased thrombocyte adhesion and aggregation.

This objective is accomplished in accordance with the invention by a medical product comprising an at least partial coating, wherein the coating comprises a functional layer and the functional layer contains saccharides and peptide sequences with integrin-binding motifs.

The invention is based on the idea of promoting cell adhesion and ingrowth on one hand, but avoiding adhesion of a particular cell type, namely thrombocyte adhesion, on the other hand. It has been found that this is possible if not only peptide sequences with integrin-binding motifs are applied to the medical product, but if saccharides are additionally integrated into the functional layer.

The inventive medical product essentially comprises at least a substrate serving as base of the actual medical product as well as a functional layer. The functional layer imparts the desired properties to the medical product.

As peptide sequence with integrin-binding motif, the RGD peptide sequence is particularly preferred, and its mediation of cell adhesion has been well studied. However, the use of other peptide sequences with integrin-binding motif is also conceivable. Where the application refers to peptides or peptide sequences, this is understood to mean peptides/peptide sequences that have at least one integrin-binding motif, unless the relevant context indicates otherwise.

In order to bring about binding of the saccharides and the peptide sequences to the surface of the medical product, preferably polymerization of the saccharides and reaction with the peptides takes place to form the functional layer. For this purpose, both the saccharides and the peptides are functionalized with polymerizable or reactive groups capable of binding to the surface of the medical product and inducing polymerization. The peptides do not necessarily have to be involved in the polymerization itself; it is sufficient if they include reactive groups through which linkage with the saccharides can be accomplished. However, the polymerizable groups of the saccharides and the reactive groups of the peptides are often chosen to be identical.

For this purpose, saccharides on the one hand and peptides on the other hand can each be bound to polymerizable/reactive groups, with a reaction, which is usually at least partially a polymerization, then taking place to form the functional layer. The polymerizable/reactive groups that are constituents of the saccharides or peptides can be identical or at least be such that a reaction can take place between them. Accordingly, copolymerization is possible, which enables reactions to take place between them. However, separate reactions of functionalized saccharides and peptides on the surface of the medical product are equally possible. To perform polymerization, a solution containing both functionalized saccharides and functionalized peptides can be applied to the substrate so that formation of the functional layer occurs from a solution. Functionalized saccharides and functionalized peptides can be present at different ratios.

However, the polymerization or the reaction of the polymerizable/reactive groups with which saccharides or peptides are provided does not have to take place simultaneously; it is also possible for a reaction of one group of substances to take place first, before reactive units of the other group of substances are then applied. It is thus a matter of two (or more) successive conversions of the various compounds endowed with polymerizable or reactive groups, namely saccharides on the one hand and peptides on the other. In this context, one also speaks of postpolymerization. In practice, the procedure is usually to first carry out a polymerization from a first solution with the first polymerizable substance group, then to switch solutions and carry out a reaction from the second solution with the second polymerizable or reactive substance group. Functionalized saccharides and functionalized peptides can also be used in varying proportions in the postpolymerization principle described.

The use of separate molecules p-saccharide on the one hand and p-peptide on the other, where p stands for any polymerizable or more generally reactive group, thus results in different variants, some of which are listed here by way of example:
a) Simultaneous polymerization of p-saccharide and p-peptide b) First polymerization of p-saccharide, followed by exchange of solutions and reaction with p-peptide
c) First reaction with or polymerization of p-peptide, followed by exchange of the solutions and reaction with or polymerization of p-saccharide
d) First polymerization of p-saccharide, followed by exchange of solutions and reaction with p-peptide, followed by another exchange of solutions and polymerization of or reaction with p-saccharide
   In a similar way, any other combinations of successive polymerizations or reactions are conceivable in principle.

Since the spatial expansion of the peptide is significantly greater than that of a monosaccharide, the amount of functionalized saccharide that usually has to be polymerized or reacted is larger than that of the functionalized peptide. This applies to a polymerization carried out at the same time as well as to successive post-polymerizations. In this way, the positive properties of the saccharide and the peptide are equally noticeable on the surface of the coated medical product. Alternatively, as described in example d) above, an additional amount of p-saccharide can be applied to deliberately increase the amount of saccharide effective on the surface.

An example of an RGD peptide that has a functional group for further reaction with polymers or polymerizable compounds is
Acetyl-Cys-Doa-Doa-Gly-Arg-Gly-Asp-Ser-Pro-NH₂ with Doa=8-Amino-3,6-dioxaoctanoic acid
   as it can be obtained from the company Cellendes GmbH, Reutlingen/Germany. Functionalization is achieved via the thiol group, via which, for example, linking with polyvinyl alcohol can take place, which in turn can be crosslinked with polyethylene glycol. The polyethylene glycol unit may also have a polymerizable acrylate function.

Another RGD peptide that can be used according to the invention is GRGDSPK (Gly-Arg-Gly-Asp-Ser-Pro-Lys), which may feature a reactive acrylate function. A coating with this peptide containing the RGD sequence, can be implemented in particular in that first a polymerization of p-saccharide is effected on the surface to be coated, followed by an exchange of the solutions for the RGD peptide solution and with the reaction with the functionalized GRGDSPK then taking place.

Another possibility is to provide polymerizable molecules carrying both the saccharide and the peptide. This can also be achieved in such a way that the polymerizable group is linked to the saccharide and the saccharide is linked to the peptide, or vice versa. When using an acrylate linker, an example would be:
Acrylate-saccharide-RGD-peptide

When polymerization takes place to form the functional layer, this automatically produces a layer that has both functionalities. However, even when polymerizable molecules with saccharide and peptide units are used, additional p-saccharide or p-peptide can be applied simultaneously, subsequently or previously to selectively influence the properties of the functional layer. As already mentioned hereinbefore, due to the steric relationships existing between peptide on the one hand and saccharide on the other hand, it is as a rule expedient to apply additional p-saccharide.

The use of polymerizable molecules carrying both the saccharide and the peptide (p-saccharide-peptide, with p standing for an arbitrary polymerizable/reactive group, irrespective of the functionality to which the polymerizable/reactive group is linked) thus results in different variants, some of which are listed hereunder as examples:
a) Simultaneous (co) polymerization of p-saccharide-peptide and p-saccharide
b) First polymerization of p-saccharide, followed by an exchange of the solutions and polymerization of p-saccharide-peptide
c) First polymerization of p-saccharide-peptide, followed by an exchange of the solutions and polymerization of p-saccharide
   A spacer may be present between the polymerizable group and the saccharide or peptide. For example, the use of a polyethylene glycol (PEG) unit is possible. The molecular weight of the PEG unit preferably ranges between 300 and 15,000 Da, in particular between 1,000 and 10,000 Da or 2,000 and 5,000 Da.

Where in the present invention reference is made to RGD peptides, this refers to peptides that have an RGD peptide sequence consisting of arginine, glycine and aspartic acid (Arg-Gly-Asp). The peptide sequence can also be present in cyclized form (CRGD). RGD peptide may be present as a tripeptide, but it may also be composed in total of a greater number of amino acids; of importance is, however, that the sequence of arginine, glycine, and aspartic acid is included, which is of significance for binding the integrins of the cells that are to adhere to the medical product. Accordingly, the RGD peptide may include, for example, the sequence
Aₚ-RGD-B_{q}
   with A and B each independently being natural or unnatural amino acids, and p and q may each be independent 0, 1, 2, 3, 4, 5, 6, 7, 8, 9, or 10. Both A and B can vary within the peptide chain.

Also possible is a cyclic peptide with an RGD sequence such as.
(—RGD-Zᵣ—),
   where again Z are identical or different, natural or unnatural amino acids and r=0, 1, 2, 3, 4, 5, 6, 7, 8, 9 or 10.

The amino acids are generally the natural L-amino acids, although as claimed by the invention the use of individual or exclusively D-amino acids is not excluded. Peptides carrying an RGD group, with amino acids or the peptide backbones being functionalized or carrying further groups, are also considered RGD peptides within the scope of the invention.

Surface coating with peptide sequences containing integrin-binding motifs is advantageously implemented in such a way that cell adherence for the endothelial cells is increased, but not that of the thrombocytes which are significantly smaller. While the size of endothelial cells ranges between about 10 and 25 μm, thrombocytes have a size of about 1 to 4 μm. In case the density of peptides serving as anchoring points is not too high, the endothelial cells, therefore, already come into sufficient peptide contact to cause a cell reaction that induces adherence, but the thrombocytes do not. For example, if the latter only come into contact with individual peptides, the mediated signal is not yet sufficient to induce thrombocyte adherence. Without wishing to be bound to any particular theory, this is presumably due to the fact that when the density of the anchor points is low, an initial adherence via the integrin receptor is still possible theoretically, but the spacings are too large for a cell activation which is mediated via integrin clustering.

From investigations that have been conducted on surfaces provided with RGD peptides, it has been found that the spacing between the immobilized peptide ligands advantageously is at least 40 nm, further advantageously at least 50 nm, in particular at least 60 nm. A spacing of the peptide ligands of up to 500 nm has been found to be particularly advantageous. Such spacing of peptide ligands on the one hand ensures sufficient attachment and adherence of endothelial cells, whereas this has not yet been observed for thrombocytes. As a result, the goal of promoting the attachment of endothelial cells and the ingrowth of a medical product, in particular an implant, is achieved without increasing the risk of thrombocyte aggregation. By appropriately selecting the reaction conditions which influence the coating reactions, such as concentration, time, temperature, selection of the functionalized peptide, etc., the desired surface coverage can be purposefully set and ascertained, for example, by means of scanning electron microscopy or atomic force microscopy.

Preferably, the saccharides linked to a polymerizable group are monosaccharides. In this context, saccharides are meant to also include reduction and oxidation products of saccharides, in particular sugar alcohols. Oligomerization or polymerization occurs only when binding to the substrate takes place. The functionalized monosaccharides are covalently bonded to the substrate, and the groups to which the monosaccharides are linked are configured such that a covalent bonding to the substrate can take place. Therefore, the functional layer comprises a complex matrix created by polymerization of the applied functionalized monosaccharides as well as reaction with the functionalized peptides.

Preferably, the saccharides in their non-functionalized form are at least to some extent sugar alcohols or relevant derivatives or isomers. Sugar alcohols (alditols) are reduction products of sugars in which an aldehyde function has been reduced to an alcohol.

A preferred sugar alcohol of the functional layer corresponds in its non-functionalized form to a sugar alcohol with the molecular formula C₆H₁₄O₆, for example sorbitol, and/or its derivatives, for example sorbitan. Other sugar alcohols can be mannitol, lactitol, xylitol, threit, erythritol or arabitol. The structure of sorbitol is shown below:

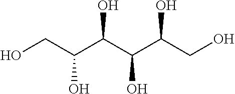 [SMILES: OC[C@@H](O)[C@@H](O)[C@H](O)[C@@H](O)CO]

“In its non-functionalized form” means that the molecular formula referred to represents the molecular formula of the non-functionalized sugar alcohol, but should also include, where appropriate, its derivatives and/or isomers thereof.

In addition to the definition of the term generally used in chemistry as “derived substance of similar structure”, and for the purposes of the invention, derivatives are to be understood as all cyclic and heterocyclic compounds derivable from the substance by dehydration. An example here is sorbitan or sorbitan anhydride, which is formed by splitting off a water molecule from sorbitol. It thus represents the anhydride of sorbitol. Another example is isosorbide, which is obtained by splitting off an additional water molecule.

The polymerizable groups via which the saccharides and peptides are functionalized may have reactive multiple bonds, especially reactive double bonds. Polymerization can thus take place via the double bonds. In particular, it may be an acrylic or methacrylic group whose suitability for polymerization reactions is known to persons skilled in the art. Other groups suitable for polymerization, such as vinyl or allyl, can also be used. With respect to the peptide, the polymerizable group, in particular a (meth)acrylate linker, may be located, for example, at the N-terminus. The oligo- or polymerization of the saccharides thus normally takes place via the polymerizable groups through which the saccharides are functionalized; on the other hand, a new formation of glycosidic bonds does not take place usually.

The solution from which the functional layer of the coating proposed by the invention is made up may thus comprise individual or several of the following substances as saccharide component:
(1) Sorbitol acrylates (composed of one or several acrylate group(s)), the acrylate group(s) of which may be located at different positions.

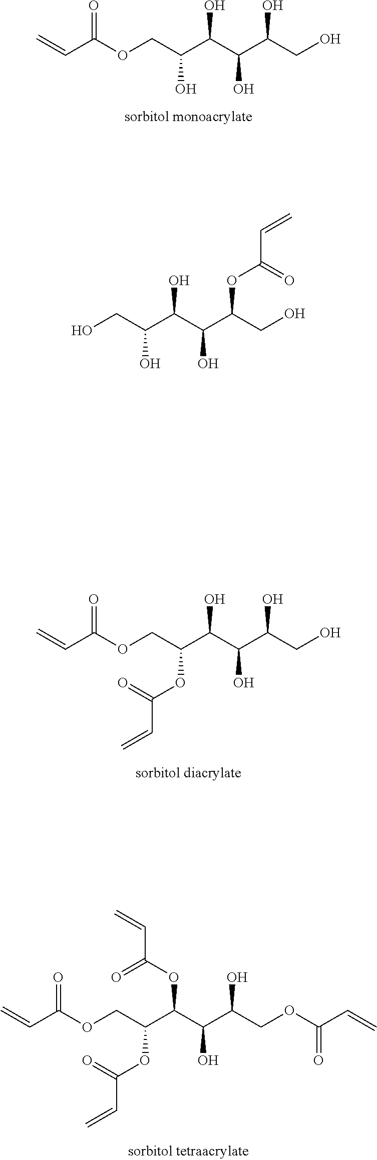 [SMILES: C=CC(=O)OC[C@@H](O)[C@@H](O)[C@H](O)[C@@H](O)CO ; C=CC(=O)O[C@@H](CO)[C@@H](O)[C@H](O)[C@H](O)CO ; C=CC(=O)OC[C@@H](OC(=O)C=C)[C@@H](O)[C@H](O)[C@@H](O)CO ; C=CC(=O)OC[C@H](O)[C@@H](O)[C@H](OC(=O)C=C)[C@@H](COC(=O)C=C)OC(=O)C=C]

(2) Sorbitol acrylates (comprising one or a multitude of acrylate group(s)), wherein the sorbitol acrylates may be partially oxidized and may comprise an aldehyde, keto and/or carboxy group.

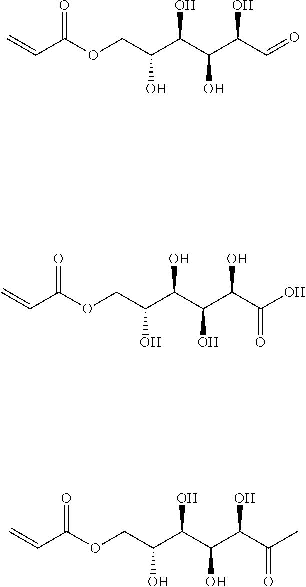 [SMILES: C=CC(=O)OC[C@@H](O)[C@@H](O)[C@H](O)[C@@H](O)C=O ; C=CC(=O)OC[C@@H](O)[C@@H](O)[C@H](O)[C@@H](O)C(=O)O ; C=CC(=O)OC[C@@H](O)[C@@H](O)[C@H](O)[C@@H](O)C(C)=O]

(3) Sorbitol acrylates (with one or a multitude of acrylate group(s)), which may comprise further reactive groups such as carboxy groups.

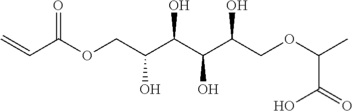 [SMILES: C=CC(=O)OC[C@@H](O)[C@@H](O)[C@H](O)[C@@H](O)COC(C)C(=O)O]

(4) Anhydrides, for example sorbitan (mono) acrylate comprising a polymerizable group.

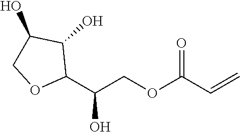

(5) Sorbitol having a non-polymerizable group, for example a carboxy group.

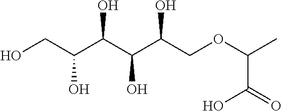 [SMILES: CC(OC[C@H](O)[C@@H](O)[C@H](O)[C@H](O)CO)C(=O)O]

(6) Complex sorbitol compounds that are not polymerizable but can be incorporated into the polymer matrix of the functional layer.

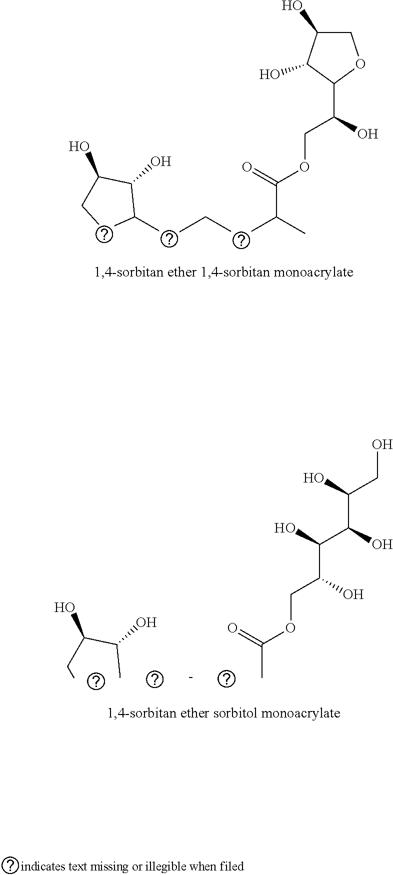

The structure of the functional layer can be varied via the specific composition of the substances. For example, it is possible to produce more tightly meshed functional layers by increasing the proportion of crosslinkers, or more lightly crosslinked functional layers with longer linear regions by using a lower proportion of crosslinkers.

In accordance with the invention, the medical product itself, which represents the substrate, is usually still covered by a carrier layer, which comprises adhesion promoters, by means of which the functional layer can be bonded to the substrate. Within the scope of the invention, preferred adhesion promoters are silane adhesion promoters. Alternatively, other adhesion promoters, for example polyolefinic adhesion promoters or adhesion promoters based on titanates or zirconates may also be employed.

Further examples of adhesion promoters include
Thiols and dithio compounds, particularly suitable for precious metal substrates
Amines and alcohols, especially suitable for platinum substrates
Carboxylic acids, especially suitable for silver substrates and aluminum substrates; the aluminum substrate may have an aluminum oxide surface
Phosphonic acids (phosphonates), especially suitable for iron, iron oxide, titanium and titanium dioxide substrates
Coordination complex building adhesion promoters, particularly chelates, which to some extent also bind non-covalently to substrates, particularly suited for various metal and metal oxide substrates

The adhesion promoters should comprise functional groups via which the adhesion promoter is capable of reacting with the functional layer, so that as a rule covalent bonding is possible. Depending on the relevant material of the medical product, bonding of the adhesion promoter to the medical product can also be covalent.

For example, a suitable adhesion promotion may be achieved by silanization, i.e., via a chemical bonding of silicon compounds, in particular silane compounds, to at least parts of their surface. On surfaces, silicon and silane compounds bind to hydroxy and carboxy groups, for example.

Silane compounds within the meaning of the invention are all those compounds which follow the general formula RₘSiXₙ (m, n=0-4, where R stands for organic radicals, in particular alkyl, alkenyl or aryl groups, and X stands for hydrolyzable groups, in particular OR, OH or halogen, with R=alkyl, alkenyl or aryl). In particular, the silane may have the general formula RSiX₃. Moreover, for the purposes of the invention relevant compounds with several silicon atoms also count among silane compounds. In particular, silane derivatives in the form of organosilicon compounds are regarded as silane compounds in accordance with the invention. Accordingly, silane compounds within the meaning of the invention are not only substances consisting of a silicon backbone and hydrogen and being designated as silanes.

Polyolefins can also be used as adhesion promoters, including chlorinated polyolefins (CPO) or acrylated polyolefins (APO).

The medical product that forms the coatable substrate can be made of a variety of materials. For example, these are metals such as nickel, titanium, platinum, iridium, gold, cobalt, chromium, aluminum, iron or alloys as well as combinations thereof. For example, a metal can also be coated with another metal, in which case the coating claimed by the invention is in turn applied to the outer metal layer, preferably comprising the carrier layer and the functional layer. Substrates in which the basic metal is covered by an oxide layer shall also count among coatable metals. Other coatable substrates are glasses.

A particularly preferred embodiment relates to a medical product that is provided in whole or in part with a gold coating through which X-ray visibility is ensured. In particular, this enables the dilation of the medical product in the blood vessel to be monitored, so that the treating physician can recognize whether the expansion is taking place as desired. This is of special advantage, for example, in the event an implant is intended for the treatment of vasospasm. The gold coating is then in turn coated with the coating of the invention, with the functional layer containing saccharides and peptide sequences with integrin-binding motifs. In most cases, a carrier layer is also applied. The base material of the medical product to which the gold coating is applied can be a customary metal or customary metal alloy intended for relevant medical products, such as a nickel-titanium alloy, a cobalt-chromium alloy, or stainless steel.

It is also possible to manufacture the medical products from various plastics, such as polyamides (PA), polytetrafluoroethylene (PTFE), expanded polytetrafluoroethylene (ePTFE), polylactides (PLA), polyester, polyether, polyurethane, polyolefins, as well as relevant block copolymers. In the field of medical engineering, those skilled in the art are familiar with a large number of suitable plastics. Whereas an adhesion promoter is usually required for metallic or oxidic surfaces, it is not always needed for polymers used as substrates.

The present invention, however, is not limited to coatings of the plastics and metals named hereinbefore, which in fact are to be understood only as examples. In principle, the invention is intended to cover coatings of all conceivable materials that are suitable for relevant medical products.

Preferably, the matrix of the functional layer is covalently bonded to the carrier layer or substrate and is preferably synthesized by graft polymerization, with the functional layer being produced on the carrier layer or substrate. The polymerization of the applied polymerizable groups carrying the saccharides and peptides preferably takes place essentially on the carrier layer/substrate, respectively, within the functional layer, and not earlier.

Different types of (graft) polymerization are understood to be included in the invention. Therefore, growth of the side chains may in particular originate from a main chain. This approach is also referred to as “grafting from”. Another possibility is that the side chains have already started with the oligomerization or polymerization and the already growing side chains are linking to the main chain (“grafting onto”). Furthermore, main and side chains that have already been oligomerized or polymerized can also congregate (“grafting through”).

Preferably, the functional layer substantially comprises a complex, highly branched, hydrophilic matrix comprising a plurality of molecules each having a main chain as polymer backbone with each having a plurality of side chains. The main and/or side chains may form bonds with other main and/or side chains. Other matrix-forming mono-, oligo- and polymers can be integrated into these main and side chains without being themselves covalently bonded to the carrier layer.

The main chain may comprise at least partially polymerized vinyl, allyl, acrylic or methacrylic compounds or derivatives thereof and/or isomers thereof or combinations thereof.

The side chains particularly comprise mono- and/or oligosaccharides, with reduction products of mono- or oligosaccharides being also understood as such, in particular sugar alcohols (alditols), as well as peptides. In addition, oxidized mono- and/or oligosaccharides may also occur, with the oxidized form also being understood as mono- or oligosaccharides for the purposes of the invention.

The medical product proposed by the invention comprises the basic structure of the medical product as substrate with a coating, said coating preferably comprising a carrier layer present on the substrate and a functional layer present on the carrier layer. The carrier layer essentially comprises the adhesion promoters, which can be covalently bonded to the substrate. In addition, non-covalently bonding adhesion promoters are also known, for example those that attach to the substrate via a coordinate bond. Preferred adhesion promoters are silicon compounds and polyolefinic adhesion promoters. According to a preferred embodiment, the functional layer comprises at least one functionalized sugar alcohol, via which the functional layer is covalently bonded to the carrier layer, as well as a functionalized peptide.

A preferred sugar alcohol of the functional layer corresponds in its non-functionalized form to a sugar alcohol of the molecular formula C₆H₁₄O₆, for example sorbitol, and/or its derivatives, for example sorbitan, and/or its isomers, such as for instance mannitol.

“In its non-functionalized form” means that the molecular formula referred to represents the molecular formula of the non-functionalized sugar alcohol, but is also intended to include, where appropriate, its derivatives and/or isomers. Functionalization shall be understood to indicate the introduction of a function into the compound permitting an attachment to the substrate, the carrier layer and/or to compounds already attached previously to the carrier layer or substrate.

The functional layer according to the invention may also comprise functionalized variants of the sugar alcohol of molecular formula C₆H₁₄O₆ and/or its derivatives and/or its isomers. The functional layer can in particular comprise a complex matrix that can be created by polymerization of the applied, functionalized sugar alcohols.

The sugar alcohols of the functional layer may at least partially be polymerized with each other.

Preferably, the coating comprises a carrier layer located on the substrate, with the functional layer in turn being bonded to the carrier layer. The bonds formed may be covalent bonds in particular, however, may also be other bonds such as coordinate bonds. The carrier layer essentially comprises the adhesion promoters bonded to the substrate. Preferred adhesion promoters are silicon compounds and polyolefinic adhesion promoters.

Without the intention of wanting to follow a particular theory, the advantage of the inventive coating with respect to the saccharides is seen in the fact that the functional layer possesses biomimetic or bio-repulsive properties and is not recognized by thrombocytes as foreign to the body, but rather as originating within the body. Accordingly, the functional layer proposed by the invention does not trigger any reaction of the thrombocytes, especially does not give rise to adhesion or aggregation reactions.

The biomimetic effect of the inventive coating is attributed to the fact that the functional layer that is claimed by the invention imitates human glycocalyx. Glycocalyx covers the cells of blood vessels with a kind of mucus layer and consists of various polysaccharides that are covalently linked to the membrane proteins (glycoproteins) and membrane lipids (glycolipids).

Of advantage regarding the biomimetic effect of the coating as proposed by the invention—and in particular of the functional layer—is that the polymerization of the reactants of the functional layer solution essentially occurs only after the functional layer solution has been applied to the substrate or carrier layer. As a result, polymerization of the reactants produces a complex layer that is so similar to the glycocalyx that the adhesion of thrombocytes to surfaces provided with the inventive coating is significantly lower than to uncoated surfaces.

The bio-repulsive effect of the coating according to the invention is based on the principle of steric repulsion. Presumably, the space available to the oligomers and polymers on the surface is reduced when a protein intrudes on this space, i.e., an approaching protein forces the oligomers and polymers on the surface to adopt an energetically less favorable conformation. This results in an overall repulsive force acting on proteins. It is also conceivable that the displacement of water molecules from the coating results in a repulsive osmotic force acting against proteins.

As regards thrombocyte/platelet adhesion, this principle of action means that the adherence of thrombocytes is prevented because there are no or only a few proteins on the surface that are suitable for bonding so that the thrombocyte adhesion is significantly reduced.

On the other hand, the presence of peptides in the functional layer causes increased adhesion of endothelial cells to the medical product, which is usually an implant intended to permanently remain in the body. The enhanced cell adherence ensures rapid endothelialization and ingrowth of the implant into the surrounding tissue.

Another advantage of the inventive coating is that, via the intermediate step of adhesion promotion, the coating covers only those surfaces and structures of the medical product which are capable of being activated for the relevant adhesion promoters and in particular have in fact been activated. Upon application of the functional layer solution, it is thus possible to place the complete medical product into the functional layer solution without having to additionally protect areas that are not to be coated.

Such a selective coating, respectively a coating process selectively carried out, offers advantages described hereinbefore for a plurality of medical products and at least for those products which are made of different materials, in which case the relevant coating is to be applied only on some of these materials.

The coating proposed by the present invention allows the coating process to activate only those parts/areas of the medical product that are intended to subsequently carry the functional layer. It is also conceivable that the medical product is already designed in such a way that the parts to be coated comprise substances that are capable of being activated for adhesion promotion.

Medical products provided with an inventive coating are suitable for endovascular, in particular neurovascular and cardiovascular fields of application as well as for peripheral areas; however, the coating thus proposed by the invention for a medical product can always be expediently applied on all medical products that come into contact with blood.

The medical product provided in accordance with the invention may be, for example, a stent (vascular endoprosthesis), such as those used for the treatment of vasoconstrictions and permanently implanted at the site of the vasoconstriction to keep the vessel open. The coating causes the implant to grow quickly into the tissue, so that after a certain time it is no longer perceived by the body as a foreign body. Typically, stents have a tubular structure and are either made by laser cutting to achieve a surface comprising struts with openings between them or they comprise a wire braiding. Stents can be delivered to the target site through a catheter and expanded there.

Stent-like implants can also be implanted in a blood vessel to treat vasospasm. Vasospasm is a spasmodic constriction of a blood vessel. Vasospasms involve the risk of blood no longer being supplied in sufficient quantities to downstream vessels (ischemia) which may lead to necrosis of the tissue thus cut off from perfusion. Placement of a vasospasm stent will cause the blood vessel to be dilated.

Another field of application for the coating is flow diverters, such as those used for the treatment of aneurysms. What has been said above with respect to the basic design and coating still applies, however, the surface coverage resp. surface density of a flow diverter typically exceeds that of a customary stent. The flow diverter is positioned in front of the neck of the aneurysm and ensures that the blood flow is diverted past the aneurysm which ensures that the aneurysm ultimately undergoes atrophy.

Another possible function a stent structure or flow diverter placed in front of an aneurysm may serve is to prevent the escape of occlusion agents such as occlusion coils incorporated into an aneurysm. Such an exit or escape of occlusion agents from the aneurysm may lead to undesirable consequences, for example resulting in occlusion agents being carried by the flow of blood to areas located further distally where they may cause the obstruction of a blood vessel or injury to the blood vessel wall. For this purpose, the stent structure can be permanently implanted in the blood vessel.

A subgroup of flow diverters are so-called bifurcation flow diverters, which are placed in front of aneurysms located at a vessel branch (bifurcation). Such a bifurcation flow diverter or bifurcation implant is described, for example, in publication WO 2014/029835 A1. The distal section of such an implant is radially expanded relative to a more proximally situated section. Said distal section has a configuration that at least partially occludes the neck of the aneurysm. The described coating to prevent thrombocyte adhesion and aggregation is also useful for such a bifurcation implant.

The terms “proximal” and “distal” are to be understood such that they refer as proximal to parts that point towards the attending physician when inserting the device, and as distal to parts that point away from the attending physician. Typically, the device is thus moved forward in distal direction with the aid of a microcatheter. The term “axial” refers to the longitudinal axis of the implant extending from proximal to distal while the term “radial” denotes levels/planes extending vertically thereto.

The implants described can be composed of webs or struts which are connected to one another. Such a structure can be fabricated by laser cutting in a basically known manner; in this context, one also speaks of cut structures. In this way, a plurality of openings or a mesh structure is created on the implant, with the openings being distributed over the circumference of the stent structure. Other manufacturing processes may be adopted as well, such as galvanic or lithographic production, 3D printing or rapid prototyping.

Alternatively, relevant implants can also be built up from a mesh structure of wires that form a braid. The wires in this case typically extend helically along the longitudinal axis, with intersecting opposed wires extending above and below each other at points of intersection resulting in honeycomb-like openings being created between the wires. The total number of wires preferably ranges between 8 and 64. As wires forming the mesh structure individual wires made of metal may be employed but it is also possible to provide strands, i.e., several wires of small diameter arranged so as to form a filament, preferably twisted around each other.

Usually at the proximal end, an implant can be connected to an insertion aid, in particular an insertion wire, via a detachment point. For example, the detachment point can be designed to be electrolytically corrodible, so that detachment of the implant occurs when an electrical voltage is applied. Likewise, other detachment points known from prior art may also be employed, in particular detachment points that can be separated mechanically, thermally or chemically. In the event of a mechanical detachment/severance, a form-, force-closed or friction fit typically exists, that is broken when the implant is liberated causing the implant to be separated from the insertion aid. In the event of a thermal detachment point, the connection can be broken by heating the detachment point, causing it to soften or melt so that severance is achieved. Another option is to make use of chemical severance in such a way that the detachment is brought about by a chemical reaction occurring at the point of detachment.

The implant can be designed to be self-expanding, i.e., it autonomously assumes an expanded state after release. For this purpose, the implant is made of materials having shape memory properties, for example nickel-titanium alloys as they are known under the name of nitinol. Other stent-like implants are dilated with the aid of a balloon, and the coating claimed by the invention can also be used for such implants.

Expediently, the medical product is provided with one or several radiopaque markers allowing the attending physician to visualize the treatment. The radiopaque markers may, for example, be made of platinum, palladium, platinum-iridium, tantalum, gold, tungsten or other metals opaque to radiation. For example, radiopaque coils may be arranged in the medical product at various points. It is also possible to provide the stent structure, in particular the struts or wires of the stent structure, with a coating comprising a radiopaque material, for example a gold coating. For example, this coating can have a thickness of between 1 and 6 μm. Coating with a radiopaque material need not cover the entire medical product; it is of particular importance in the areas that are in contact with the inner vessel wall, i.e., essentially in the cylindrical portion of a stent structure. Nevertheless, even when applying a radiopaque coating it is considered useful to arrange one or several radiopaque markings, in particular at the distal end of the medical product.

In addition to the inventive medical product, the invention also relates to a method for manufacturing such a medical product, namely a method for coating a medical product with a functional layer, with the functional layer being produced by reacting saccharides functionalized with reactive groups and peptides functionalized with reactive groups, with the reaction as a rule being at least partially a polymerization.

All the explanations and described combinations of features relating to the medical product also apply in a corresponding manner to the inventive method and vice versa.

It is to be noted that any features and characteristics individually included in the claims may also be combined with each other in an optional and technologically sensible manner so that they reveal further implementations or methods of the invention.

### Tests

Series of experiments were performed in vitro with the coating proposed by the invention to investigate the influence on adhesion of endothelial cells on the one hand and thrombocytes on the other hand. The small nitinol sample plates examined were divided into 3 groups:
1) Uncoated small nitinol plates (A+D)
2) Small nitinol plates with a coating of sugar alcohols polymerizing on the surface and functionalized with polymerizable groups (B+E).
3) Small nitinol plates corresponding to the 2nd group that underwent additional coating with the peptide GRGDSPK (C+F)
   The results are shown in FIG. 1. Plates A to C were seeded with human endothelial cells (HUVEC; human umbilical vein endothelial cells) and stained with a specific fluorescent dye. Plates D to F were incubated with human whole blood. Adherent thrombocytes were stained with a specific fluorescent dye.

It can be seen that the uncoated plates A and D show a strong accumulation of cells, both endothelial cells (A) and thrombocytes (D). In contrast, plates B and E, in which only a saccharide layer was applied, show almost no attachment of cells, neither endothelial cells (B) nor thrombocytes (E). On the one hand, the absence of thrombocytes is positive to prevent renewed thrombus formation; on the other hand, adherence of endothelial cells to promote the ingrowth of an implant would be desirable.

The latter is shown by plates C and F. Endothelial cell colonization (C) is strong and of a magnitude which is comparable to the uncoated plates. The accumulation of thrombocytes, on the other hand, remains negligible (F).

1. A medical product, wherein at least portions of the medical product are provided with a coating and said coating comprises a functional layer, wherein:
the functional layer contains saccharides and peptide sequences with integrin-binding motifs.

2. A medical product according to claim 1, wherein the functional layer comprises an RGD peptide sequence.

3. A medical product according to claim 1, wherein the functional layer is created by a reaction of saccharides functionalized with polymerizable groups and peptides functionalized with reactive groups with integrin-binding motifs.

4. A medical product according to claim 3, wherein the functional layer is created by a copolymerization of saccharides functionalized with polymerizable groups and peptides functionalized with reactive groups with integrin-binding motifs.

5. A medical product according to claim 3, wherein the functional layer is created by successive reactions of saccharides functionalized with polymerizable groups and peptides functionalized with reactive groups with integrin-binding motifs.

6. A medical product according to claim 3, wherein the functional layer is created by successive reactions of peptides functionalized with reactive groups with integrin-binding motifs and with saccharides functionalized with polymerizable groups.

7. A medical product according to claim 3, wherein the functional layer is created by a polymerization of polymerizable molecules carrying a saccharide unit and a peptide unit, with the peptide having at least one integrin-binding motif.

8. A medical product according to claim 1, wherein the saccharides are monosaccharides.

9. A medical product according to claim 1, wherein the saccharides in their non-functionalized form are at least partially sugar alcohols or cyclic or heterocyclic compounds derivable from sugar alcohols by dehydratization.

10. A medical product according to claim 1, wherein the polymerizable groups comprise reactive multiple bonds.

11. (canceled)

12. A medical product according to claim 1, wherein the coating comprises a carrier layer with an adhesion promoter, and the functional layer is bonded to the carrier layer.

13. A medical product according to claim 12, wherein the bond of the adhesion promoter to the medical product and/or or the bond of the functional layer to the carrier layer is a covalent bond.

14. A medical product according to claim 12 wherein the adhesion promoter comprises a silicon compound.

15. A medical product according to, claim 1, wherein a spacer is present between the polymerizable group and the saccharide or peptide.

16. A medical product according to claim 1, wherein the medical product is at least partially made of a metal selected from the group comprising nickel, titanium, platinum, iridium, gold, cobalt, chromium, aluminum, iron or an alloy of the aforementioned metals.

17. A medical product according to claim 1, wherein the medical product is at least partially made of a plastic material selected from the group comprising polyamides (PA), polytetrafluoroethylene (PTFE), expanded polytetrafluoroethylene (ePTFE), polylactides (PLA), polyesters, polyethers, polyurethanes, polyolefins and corresponding block copolymers.

18. A medical product according to claim 1, wherein the medical product is an implant intended to remain permanently in the body.

19. A medical product according to, claim 1 wherein the medical product is an endovascular medical product.

20. A medical product according to claim 19, wherein the medical product is a neurovascular or cardiovascular medical product or a medical product for the peripheral area.

21. A method of coating a medical product with a functional layer, wherein the functional layer is produced by the reaction of saccharides functionalized with reactive groups and peptides functionalized with reactive groups with integrin-binding motifs.

22. A medical product according to claim 10, wherein the reactive multiple bonds are reactive double bonds.

23. A medical product according to claim 14, wherein the adhesion promoter comprises a silane compound.

24. A medical product according to claim 22, wherein at least one of the reactive double bond is a constituent of a (meth)acrylic, allyl or vinyl group.
